(19) United States
(12) Patent Application Publication
(10) Pub. No.: US 2022/0143356 A1
(43) Pub. Date: May 12, 2022

(54) DEW POINT SENSOR SYSTEM, METHOD FOR MODULATING A HUMIDIFIER HEATER PLATE, AND HUMIDIFIER/MEDICAL DEVICE CONTAINING

(71) Applicants: VINCENT MEDICAL (DONG GUOAN) MANUFACTURING CO., LTD., Guangdong (CN); VINCENT MEDICAL (DONGGUAN) TECHNOLOGY CO., LTD., Dobgguan, Guangdong (CN)

(72) Inventors: Jiebing XU, Guangdong (CN); Haibin YU, Guangdong (CN); Jun ZHAO, Guangdong (CN); Wei ZHU, Guangdong (CN)

(21) Appl. No.: 17/437,513

(22) PCT Filed: Jun. 6, 2019
(86) PCT No.: PCT/CN2019/090385

§ 371 Date: Sep. 9, 2021

(51) Int. Cl. A61M 16/16; A61M 16/00; A61M 16/10

(52) U.S. Cl. CPC A61M 16/161; A61M 16/024; A61M 16/109; A61M 16/0066; A61M 2205/3379; A61M 2016/003; A61M 2205/18; A61M 2205/3334; A61M 2205/3368; A61M 16/0003

(57) ABSTRACT

Dew point sensor system contains-a controller with dew point calculator, ambient temperature sensor, air pathway, heater plate, external wire. Ambient temperature sensor generates ambient temperature data, operatively-connected to controller. Air pathway contains blower within air pathway, air flow sensor within air pathway, liquid reservoir operatively-connected to air pathway, and component may be air pathway temperature sensor and/or breathing circuit heating element. Blower causes air to move in air pathway (52), air flow sensor generates air flow data about air moving in air pathway. The heater plate contains heater plate temperature sensor, and heater plate temperature sensor generates heater plate temperature data. External wire is operatively-connected to controller and component. Ambient temperature sensor located on the external wire. Heater plate and the heater plate temperature sensor operatively-connected to the controller receiving ambient temperature data, air flow data, and heater plate temperature data to calculate target heater plate temperature, based on dew point temperature.

### FIELD OF THE INVENTION

The present invention relates to a dew point sensor system and a machine, such as a humidifier, containing such a dew point sensor system. More specifically, the present invention relates to a dew point sensor system containing an ambient temperature sensor.

### BACKGROUND

Humidifiers are used to provide humidified air to a user, typically patients in hospitals, hospices, and even at home. Humidifiers are often operated in a variety of conditions, ranging at temperatures of from about 10° C. to about 40° C. and above. It is well-known that the amount of water potentially held in the air is dependent on the temperature of the air, and thus there is a distinction between relative humidity and absolute humidity.

Accordingly, humidifiers which measure the ambient air temperature are known as seen in, for example, U.S. Pat. No. 8,616,202 B2 to Fisher & Paykel Healthcare, Ltd., published on Dec. 31, 2013; and U.S. Pat. No. 10,058,663 B2 to Fisher & Paykel Healthcare, Ltd., published on Aug. 28, 2018.

Such references typically include an ambient temperature sensor within the housing of the device and/or attached to the housing of the device, such as, for example, on the external surface of the air passageway. However, it has now been found that such ambient temperature sensor locations do not provide accurate temperature readings of the ambient temperature, which can then adversely-affect the calculations of the dew point and cause the humidifier to provide an inaccurate amount of heating.

Accordingly, it is desirable to provide a humidifier having an ambient temperature sensor which more accurately measures the ambient temperature. In addition, it is desirable to provide a humidifier and/or a medical device with a more accurate ambient temperature sensor.

### SUMMARY OF THE INVENTION

An embodiment of the present invention relates to a dew point sensor system containing a controller with a dew point calculator, an ambient temperature sensor, an air pathway, a heater plate, and an external wire. The ambient temperature sensor generates ambient temperature data, the ambient temperature sensor operatively-connected to the controller. The air pathway contains a blower within the air pathway, an air flow sensor within the air pathway, a liquid reservoir operatively-connected to the air pathway, and a component which may be an air pathway temperature sensor and/or a breathing circuit heating element. The blower causes air to move in the air pathway, while the air flow sensor generates air flow data about the air moving in the air pathway. The heater plate contains a heater plate temperature sensor, and the heater plate temperature sensor generates heater plate temperature data. An external wire operatively-connecting the controller and the component.

The ambient temperature sensor is located on the external wire. The heater plate, and the heater plate temperature sensor are operatively-connected to the controller. The controller references a dew point temperature and receives the ambient temperature data, the air flow data, and the dew point temperature to calculate the target heater plate temperature.

In an embodiment of the invention herein, a method for modulating a humidifier heater plate contains the steps of A) providing a humidifier comprising the dew point sensor system herein, B) generating ambient temperature data; C) transmitting the ambient temperature data to the controller; D) generating air flow data; E) transmitting the air flow data to the controller; F) generating heater plate temperature data; G) transmitting the heater plate temperature data to the controller; H) referencing a dew point temperature; I) transmitting the required dew point temperature to the controller; J) receiving the ambient temperature data, the air flow data, and the dew point temperature at the controller; K) calculating a target heater plate temperature based on the ambient temperature data, the air flow data, and the dew point temperature; L) comparing the target heater plate temperature with the heater plate temperature data and M) the controller adjusting the heater plate temperature to the target heater plate temperature based on the target heater plate temperature and the heater plate temperature data.

In an embodiment of the present invention, a humidifier may contain the dew point sensor system herein.

In an embodiment of the present invention, a medical device may contain the dew point sensor system herein.

Without intending to be limited by theory, it is believed that the present invention may provide significantly more accurate dew point calculations, as compared to systems which contain an ambient temperature sensor which is within, and/or immediately connected-to the housing. As mentioned, even if the ambient temperature sensor is connected to the air pathway external to the housing, the air passing through the air pathway is typically already heated in some manner. Accordingly, the ambient temperature sensor of the present invention, which is attached to the external wire, is better situated to accurately-measure the ambient temperature. This better, more accurate ambient temperature data in turn may lead to, for example, a more accurate calculation of the real-time dew point, more accurate management of the heater plate temperature, etc.

### BRIEF DESCRIPTION OF THE DRAWINGS

FIG. 1 shows a schematic view of an embodiment of the electronic components of the dew point sensor system;

FIG. 2 shows an embodiment of a humidifier of the present invention; and

FIG. 3 shows an embodiment of the invention herein containing a blower.

The figures herein are for illustrative purposes only and are not necessarily drawn to scale.

### DESCRIPTION OF THE PREFERRED EMBODIMENTS

Unless otherwise specifically provided, all measurements are made in metric units. Furthermore, all percentages, ratios, etc. herein are by weight, unless specifically indicated otherwise.

As used herein the term “operatively-connected” indicates that the item(s) is(are) connected in a manner which allows them to operate. This may involve, for example, wires, a transmitter/receiver pair, a pair of transceivers, etc. This phrase may also indicate that a physical structure connects the various indicated items.

As used herein, the term “user” indicates the user of the invention herein, and may be, for example, an owner, a patient, a car-giver, etc.

Unless otherwise specifically described all of the devices, items and/or parts herein may be made from industry-standard materials which are available from multiple suppliers worldwide.

In an embodiment of the present invention, a dew point sensor system contains a controller, an ambient temperature sensor, an air pathway, a heater plate. The controller contains a dew point calculator, and the ambient temperature sensor generates ambient temperature data. The ambient temperature sensor is operatively-connected to the controller. The ait pathway contains a blower, a flow sensor within the air pathway, a liquid reservoir operatively-connected to the air pathway, and component which is selected from an air pathway temperature sensor, a breathing circuit heating element, and a combination hereof. The blower causes the air to move in the air pathway, and the flow sensor generates air flow data from the air moving in the air pathway. The heater plate is operatively-connected to heat the liquid reservoir. The heater plate also contains a heater plate temperature sensor which measures the heater plate temperature and generates heater plate temperature data. An external wire is operatively-connected to the controller and the component.

In an embodiment herein, the user sets the desired air flow and the controller adjusts the blower accordingly. In an embodiment herein, if the air flow data shows that the air flow is different; or at least 5% different; or at least 10% different, from the desired air flow set by the user, then the controller may sound; or sounds, an alarm. In an embodiment herein, the controller sounds an alarm only if the difference between the air flow in the air pathway and the desired air flow exists for a continuous period of time of about ≥30 seconds; or about ≥1 minute; or about ≥2 minutes.

The ambient temperature sensor is located on the external wire. The heater plate and the heater plate temperature sensor are both operatively-connected to the controller. The controller references a dew point temperature. The controller also receives the ambient temperature data, the air flow data, and the dew point temperature to calculate the target heater plate temperature. The blower is optionally operatively-connected to the controller. The controller may modulate the heater plate temperature by, for example, increasing the heater plate temperature, decreasing the heater plate temperature, or both.

The controller may reference the dew point temperature by, for example, referring to a look-up table, calculating the dew point temperature, etc. as desired.

Without intending to be limited by theory, it has surprisingly-been found that an ambient temperature sensor connected within the housing of a humidifier within the air pathway is typically reading an inaccurate temperature. Even when the ambient temperature sensor is located in, or adjacent-to, the air pathway before the liquid chamber/humidification chamber, it has been found that the temperature within the housing itself and/or the surface of the housing, and the air pathway connected thereto are often warmer than the ambient temperature because of the heat radiated by the blower, the heater plate, the internal electronics and power system, etc. and the temperature sensed may be higher, or even significantly-higher, for example, up to 10° C. higher, than the actual ambient temperature. Surprisingly, it has been found that even when the ambient temperature sensor is connected to the external surface of the air pathway, the temperature sensed is often higher than the actual ambient temperature.

Such an inaccurate ambient temperature data would of course, then adversely-affect the controller's dew point calculation, and therefore could cause, for example, increased condensation in the air pathway, etc.

Accordingly, in an embodiment herein, the ambient temperature sensor is affixed to the external wire at a position external to the housing, so as to sense the ambient temperature of the air which is being drawn into the housing and therefore the air pathway, by the blower. In an embodiment herein, the external wire does not conduct electricity to or from any heating elements within, external to, or adjacent to the air pathway. Without intending to be limited by theory it is believed that a wire which conducts electricity to or from a heating element, such as those within, external to, or adjacent to an air pathway, may also become warm and therefore throw off the ambient temperature data generated by the ambient temperature sensor.

In an embodiment herein, the dew point sensor system is further contains a housing. The housing typically contains, or encompasses the air pathway, the heater plate, and the controller. The liquid reservoir may be partially-contained within the housing, affixable to the housing, etc. and typically the liquid reservoir directly-contacts the heater plate.

In an embodiment herein, the air pathway temperature sensor is located within the air pathway downstream of the liquid reservoir. Alternatively, the air pathway temperature sensor is located within the air pathway immediately downstream of the liquid reservoir. Alternatively, the air pathway temperature sensor is located downstream of the liquid reservoir and is proximate to; or immediately upstream of, the user.

In an embodiment herein, the controller employs; or contains; or references, a dew point calculator to calculate the dew point from the ambient temperature data, the air flow data, and the heater plate temperature data. In an embodiment herein, the dew point calculator is a look-up table to calculate the dew point temperature from the ambient temperature data, the air flow data, and the heater plate temperature data. Typically, the dew point temperature is calculated as a function of the air flow rate within the air pathway, the heater plate temperature, and the ambient (room) temperature. So, for a given flow rate set by the user (i.e., “desired air flow”), ambient temperature, and a given dew point temperature set by the user; the controller can calculate the target heater plate temperature and the controller then adjusts the power flowing to the heater plate to achieve this temperature. In an embodiment herein, some data; or all data, is real-time data. In an embodiment herein, the controller adjusts the power flowing to the heater plate if the difference between the heater plate temperature and the target heater plate temperature is about >0.1° C. In such an embodiment, if the difference between the heater plate temperature and the target heater plate temperature is about ≤0.1° C., then the controller does not adjust the power flowing to the heater plate.

In an embodiment herein, it may be desirable for the dew point system herein to contain multiple; or a plurality of air pathway temperature sensors. In an embodiment herein, a liquid reservoir-side air pathway temperature sensor is located within the air pathway and downstream; or immediately downstream, of the liquid reservoir, and a patient-side air pathway temperature sensor is located within the air pathway downstream of the liquid reservoir and proximate to; or immediately upstream of, the user.

The dew point is also dependent upon the speed of the air flowing in the air pathway, and therefore the dew point system herein contains a blower to move air through the air pathway and an air flow sensor to generate air flow data from the air moving in the air pathway. In an embodiment herein, the desired air flow (rate) is set at a specific speed by the user and then the controller adjusts the blower accordingly, so that this is removed as a variable—in effect, this is a constant air flow value that is stabilized by the controller. In an embodiment herein, the blower has a blower operating speed and the controller may reference; or references, the air flow data to modulate the blower operating speed. The controller may modulate the blower operating speed by, for example, increasing the blower operating speed, decreasing the blower operating speed, or both.

In an embodiment herein, due to the importance of receiving accurate heater plate temperature data, the heater plate temperature sensor is directly-affixed to the heater plate. Without intending to be limited by theory, it is believed that such an arrangement allows for a direct measurement of the heater plate temperature, instead of, for example, an estimation of the heater plate temperature via a measurement of the electrical load being used to heat up the heater plate.

In addition to modulating the heater plate temperature with respect to the dew point, the controller may also modulate the hear plate temperature in response to when the heater plate temperature data indicates an unacceptably high temperature, such as above about 120° C.; or above about 150° C., etc. Such data would indicate that the water reservoir has run dry and therefore the heater plate should be shut off so as to avoid damage to the heater plate, the liquid reservoir, etc.

The liquid reservoir herein is typically a transparent or translucent container which is made of heat-resistant materials such as high density plastic, glass, metal, and a combination thereof; or polypropylene, polycarbonate, aluminium, and a combination thereof; or polycarbonate, aluminium, and a combination thereof. The liquid reservoir contains the liquid; or water; or distilled water; or sterile water, which is evaporated to create the humidified air. The liquid reservoir is operatively-connected; or fluidly-connected, to the air pathway so as to allow the typically can be opened so as to allow the user (or another person) to add a liquid; or water; or distilled water; or sterile water, thereto.

In an embodiment herein, the dew point sensor system may further contain an; or a plurality of, air pathway humidity sensor located within the air pathway which generate air pathway humidity data and transmits such air pathway humidity data to the controller.

In an embodiment herein, a humidifier contains the dew point sensor system described herein. In an embodiment herein, the dew point sensor system herein is contained within, or as a part of a medical device.

The dew point sensor system, humidifier, and/or medical device herein is typically operatively-connected to a power system such as a power supply, a battery, a power grid, an AC power outlet, etc.

Method

In an embodiment herein, a method for modulating the dew point of a humidifier contains the steps of providing a humidifier containing a dew point sensor system, generating ambient temperature data, transmitting the ambient temperature data to the controller, generating air flow data, transmitting the air flow data to the controller, generating heater plate temperature data, transmitting the heater plate temperature data to the controller, referencing a dew point temperature, transmitting the dew point temperature to the controller, receiving the ambient temperature data, the air flow data, the heater plate temperature data and the dew point temperature at; or by, the controller, calculating a target heater temperature based on the ambient temperature data, the air flow data, and the dew point temperature, and comparing the target heater plate temperature with the heater plate temperature data.

The controller calculates the target heater temperature based on the ambient temperature data, the air flow data (set by user), and a set dew point temperature. If the heater plate temperature data shows that the heater plate is lower than the target heater plate temperature, then the controller adjusts the heater plate temperature to the target heater plate temperature based on the target heater plate temperature and the heater plate temperature data. In an embodiment herein, the controller adjusts the heater plate temperature if the difference between the heater plate temperature and the target heater plate temperature is about ≥0.1° C.; or about ≥2° C.; or about ≥4° C. In an embodiment herein, if the controller finds that the heater plate temperature is higher than the target heater plate temperature, then the controller may adjust the heater plate temperature by decreasing the power flowing to the heater plate. If the controller finds that the heater plate temperature is lower than the target heater plate temperature, then the controller may adjust the heater plate temperature by increasing the power flowing to the heater plate. Alternatively, in either of the above cases, the controller may sound an alarm.

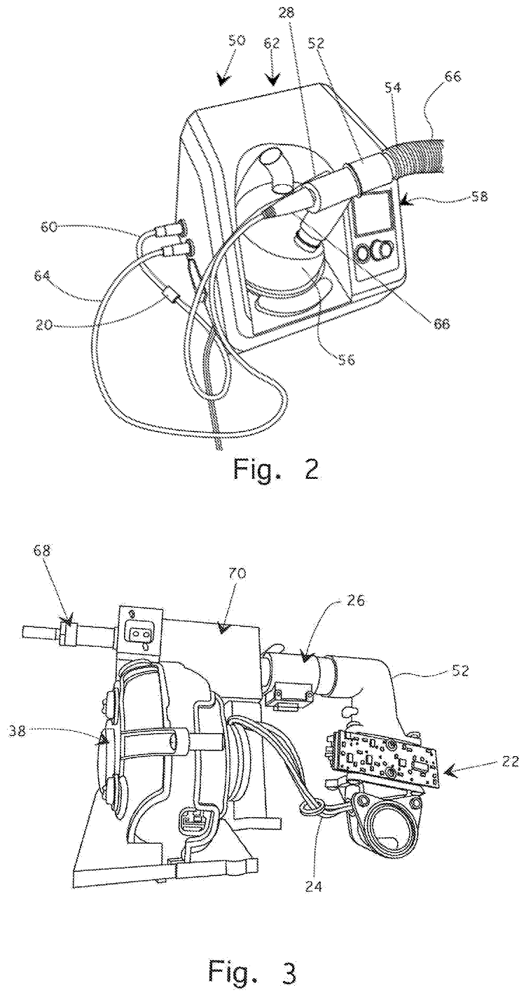

In an embodiment of the method herein, the dew point sensor system may further contain a breathing circuit (see FIG. 2 at 54), the breathing circuit comprises a heating circuit to heat the breathing gas. the dew point sensor system may further contain an air pathway temperature sensor which provides air pathway temperature data to the controller, and where the method may further include the step of the controller adjusting the power flowing to the heating circuit based upon the air pathway temperature data. The various types of data described herein (e.g., ambient temperature data, air flow data, heater plate temperature data, air pathway temperature data, air pathway humidity data, etc.) may be transmitted to the controller either via wires, or via, for example, radio or other wireless signals.

In an embodiment herein, the controller further references; or calculates, the real-time dew point temperature based on the heater plate temperature data, the air flow data, and the ambient temperature data. In an embodiment herein, if the controller discovers that the difference between the set dew point temperature data and the real-time dew point temperature is about ≥1° C.; or about ≥2° C.; or about ≥4° C. after warm-up (about 20 minutes after the heater plate is activated), then the controller sounds an alarm. In an embodiment of the invention herein, if the controller discovers that the difference between the set dew point temperature data and the real-time dew point temperature is about ≥1° C.; or about ≥2° C.; or about ≥4° C. after warm-up (about 20 minutes after the heater plate is activated) for a continuous period of time of about ≥30 seconds; or about ≥1 minute; or about ≥2 minutes, then the controller sounds an alarm.

In an embodiment herein, the alarm herein may be a notification, such as a sound, a light, a computer message, a wireless signal, and/or any other signal intended to notify and/or draw the attention of the user.

In an embodiment herein, the process further contains the step of repeating the steps from the generating of the ambient temperature data to the step of the controller adjusting the heater plate temperature to the target temperature based on the target temperature and the heater plate temperature data.

In an embodiment herein, the dew point sensor system herein generates ambient temperature data and sends this data to the controller. The blower starts up and air flow data is generated and transmitted to the controller. In an embodiment herein, the user sets a desired air flow and the controller adjusts the blower speed to provide the desired air flow. In an embodiment herein, if the air flow data shows that the air flow is different; or at least 5% different; or at least 10% different, from the desired air flow set by the user, then the controller may sound an alarm. The heater plate is activated by the controller and the heater plate temperature sensor generates heater plate temperature data which is transmitted to the controller. The controller references; or calculates, the target heater plate temperature based on the ambient temperature data, the air flow data and the set dew point temperature.

The controller then compares the heater plate temperature data with the target heater plate temperature. If the heater plate temperature data indicates the difference between the heater plate temperature and the target heater plate temperature is about ≥0.1° C.; or about ≥2° C. lower; or about ≥4° C., then the controller adjusts the heater plate temperature by appropriately increasing or decreasing the heater plate temperature; or appropriately increasing or decreasing the power flowing to the heater plate.

In an embodiment of the method herein, the controller checks the system at a rate of at least about 600 times/minute; or from about 1 times/minute to about 1000 times/minute; or from about 10 times/minute to about 800 times/minute.

In an embodiment herein, after turning on the humidifier and/or medical device, the heater plate warms up for a predetermined period of time; or from about 1 minute to 35 minutes; or from about 2 minutes to about 30 minutes. The heater plate temperature data is generated in step (F) when the heater plate is activated.

In an embodiment herein, the medical device is selected from the group of a humidifier, a continuous positive air pressure machine, an automatic positive air pressure machine, and a combination thereof; or a humidifier.

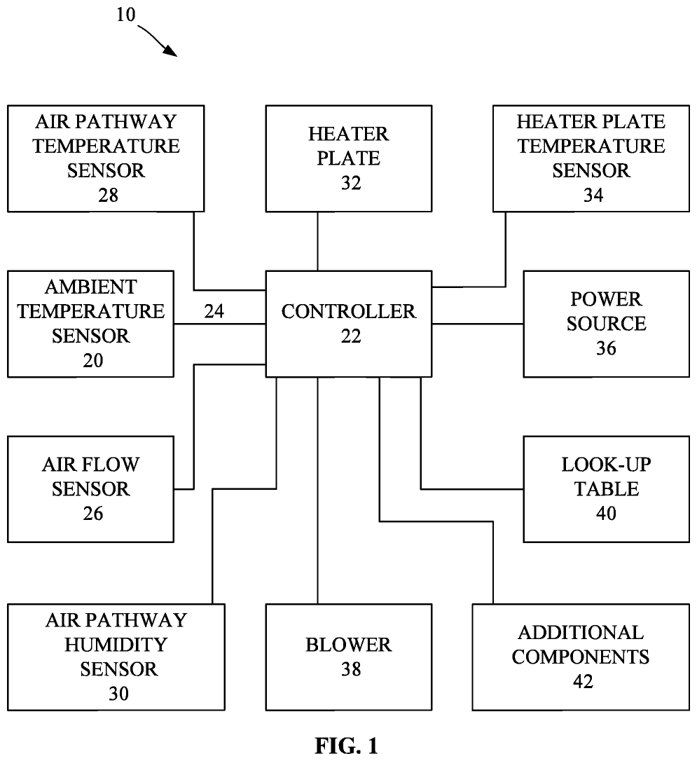

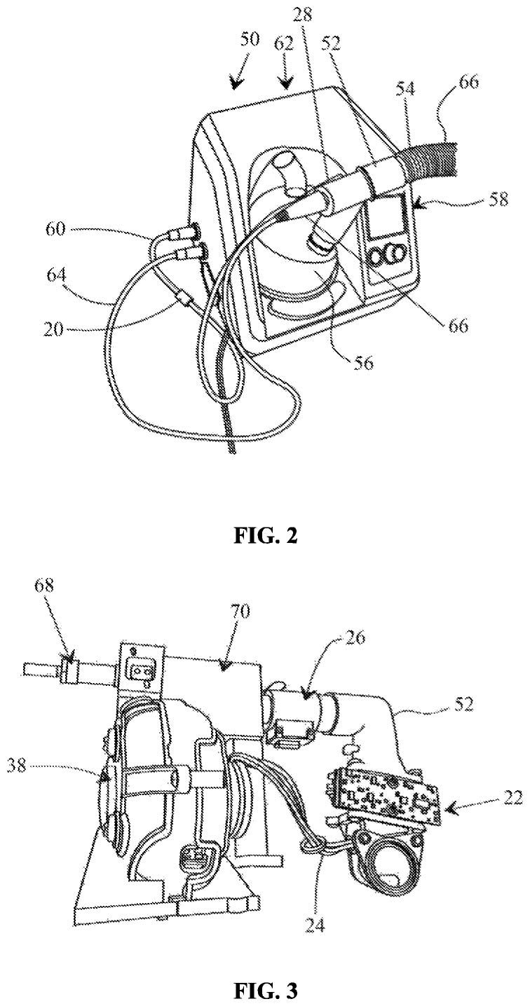

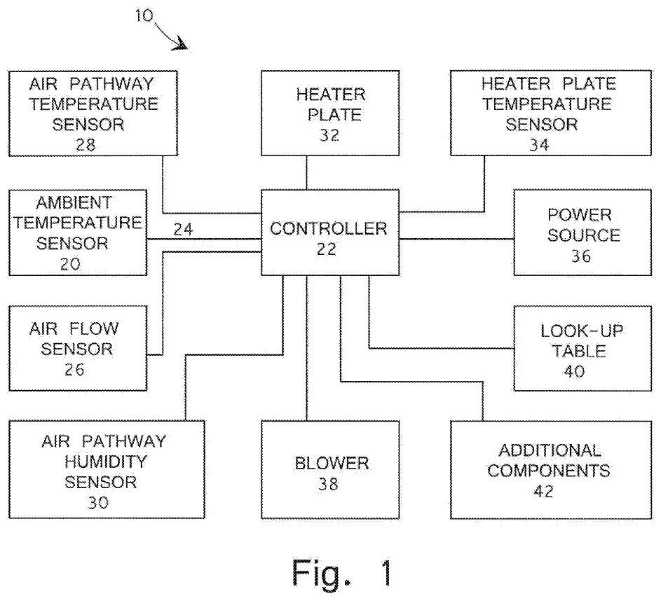

Turning to the figures, FIG. 1 shows a schematic view of an embodiment of the electronic components of the dew point sensor system, 10. An ambient temperature sensor, 20, is operatively-connected to a controller, 22, by a wire, 24. The ambient temperature sensor generates ambient temperature data and transmits it to the controller via the wire. An air flow sensor, 26, is also operatively-connected to the controller, 22, generates air flow data, and transmits it to the controller, 22. An air pathway temperature sensor, 28, is operatively-connected to the controller, 22, generates air pathway temperature data, and transmits it to the controller, 22. Also in FIG. 1, an air pathway humidity sensor, 30, is operatively-connected to the controller, 22, generates air pathway humidity data, and transmits it to the controller, 22.

A heater plate, 32, is operatively-connected to the controller, 22, as is a heater plate temperature sensor, 34. The controller, 22, modulates the heater plate temperature by adjusting the amount of electricity powering the heater plate, 32. The controller, 22, is also operatively-connected to a power source, 36, which delivers electrical power to the controller, 22. A blower, 38, is operatively-connected to the controller, 22. The controller, 22, modulates the blower operational speed by, for example, adjusting the amount of electricity provided to the blower, 38.

The controller, 22, is also operatively-connected to a look-up table, 40, which is used by the controller, 22, to calculate the target heater plate temperature and the dew point temperature. The controller, 22, may also be connected to one or more additional components, 42, which may provide additional functionality, such as a clock, an alarm, a water level sensor, a heating circuit for the breathing circuit, and a combination thereof.

FIG. 2 shows an embodiment of a humidifier, 50, of the present invention. An air pathway, 52, contains a breathing circuit, 54, a liquid reservoir, 56, a control panel, 58, and an external wire, 60, which is outside of a housing, 62, which contains, for example, the controller (see FIG. 1 at 22), the heater plate (see FIG. 1 at 32), the heater plate temperature sensor (see FIG. 1 at 34), the blower (see FIG. 1 at 36), etc. The ambient temperature sensor, 20, is located; i.e., attached to, the external wire, 60.

The embodiment of FIG. 2 also shows an optional heating circuit wire, 64, which provides electricity to a heating circuit, 66, located within the breathing circuit, 54. The external wire, 60, is connected to the air pathway temperature sensor, 28, which monitors the temperature at the humidifier-side of the breathing circuit, 54. The air pathway temperature sensor, 28, could also be located at the patient-end of the breathing circuit, 54.

FIG. 3 shows a side view of an embodiment of the blower, 38, herein. FIG. 2 shows the blower connected to part of the air pathway, 52, which contains the air flow sensor, 26, therein. FIG. 3 also shows an oxygen inlet, 68, upstream of a mixing chamber, 70, which combines and homogenizes air from the blower, 38, and oxygen from the oxygen inlet, 68. The controller, 22, is operatively-connected to the blower, by the wires, 24.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

1. A dew point sensor system comprising:
A) a controller comprising a dew point calculator;
B) an ambient temperature sensor which generates ambient temperature data, the ambient temperature sensor operatively connected to the controller;
C) an air pathway comprising; i) a blower within the air pathway, wherein the blower causes air to move in the air pathway; ii) an air flow sensor within the air pathway, wherein the air flow sensor generates air flow data about the air moving in the air pathway; iii) a liquid reservoir operatively connected to the air pathway; and iv) a component selected from the group consisting of an air pathway temperature sensor, a breathing circuit heating element, and a combination thereof;
D) a heater plate operatively connected to heat the liquid reservoir, wherein the heater plate comprises a heater plate temperature sensor, and wherein the heater plate temperature sensor generates heater plate temperature data; and
E) an external wire operatively-connecting the controller and the component, wherein the ambient temperature sensor is located on the external wire, wherein the heater plate, and the heater plate temperature sensor are operatively-connected to the controller, and optionally where the blower is operatively connected to the controller, wherein the controller references a dew point temperature, and wherein the controller receives the ambient temperature data, the air flow data, and the dew point temperature to calculate the target heater plate temperature.

2. The dew point sensor system according to claim 1, wherein the air pathway temperature sensor is located in the air pathway outlet downstream of the liquid reservoir; or wherein the air pathway temperature sensor is located immediately downstream of the liquid reservoir; or wherein the air pathway temperature sensor is located downstream of the liquid reservoir and proximate to a user.

3. The dew point sensor system according to claim 1, comprising a plurality of air pathway temperature sensors.

4. The dew point sensor system according to claim 1, further comprising an air pathway humidity sensor downstream of the liquid reservoir; or downstream of the liquid reservoir and proximate to a user.

5. The dew point sensor system according to claim 1, wherein the component is the air pathway temperature sensor.

6. The dew point sensor system according to claim 1, wherein the heater plate temperature sensor is directly-affixed to the heater plate.

7. The dew point sensor system according to claim 1, wherein the liquid reservoir comprises a liquid; or water; or distilled water; or sterile water.

8. The dew point sensor system according to claim 1, wherein the controller employs a look-up table to calculate the target heater plate temperature and/or dew point.

9. The dew point sensor system according to claim 1, further comprising a housing, wherein the housing comprises; or contains, the air pathway, the heater plate and the controller.

10. A method for modulating a humidifier heater plate comprising the steps of:
A) providing a humidifier comprising the dew point sensor system according to any one of the previous claims;
B) generating ambient temperature data;
C) transmitting the ambient temperature data to the controller;
D) generating air flow data;
E) transmitting the air flow data to the controller;
F) generating heater plate temperature data;
G) transmitting the heater plate temperature data to the controller;
H) referencing a dew point temperature;
I) transmitting the dew point temperature to the controller;
J) receiving the ambient temperature data, the air flow data, and the dew point temperature at the controller;
K) calculating a target heater plate temperature based on the ambient temperature data, the air flow data, and the dew point temperature;
L) comparing the target heater plate temperature with the heater plate temperature data; and
M) adjusting the heater plate temperature via the controller to the target heater plate temperature based on the target heater plate temperature and the heater plate temperature data.

11. The method for modulating the dew point of a humidifier according to claim 10, wherein the dew point temperature is a set dew point temperature.

12. The method for modulating the dew point of a humidifier according to claim 11, wherein the controller further calculates a real-time dew point temperature based on the heater plate temperature data, the air flow data, and the ambient temperature data.

13. The method for modulating the dew point of a humidifier according to claim 10, further comprising the step of activating the heater plate, and wherein the generating of heater plate temperature data in step (F) occurs after the heater plate is activated.

14. The method for modulating the dew point of a humidifier according to claim 12, wherein if the controller discovers that the difference between the set dew point temperature data and the real-time dew point temperature is about ≥1° C.; or about ≥2° C.; or about ≥4° C. after warm-up, then the controller sounds an alarm.

15. The method for modulating the dew point of a humidifier according to claim 14, wherein if the controller discovers that the difference between the set dew point temperature data and the real-time dew point temperature is about ≥1° C.; or about ≥2° C.; or about ≥4° C. after warm-up for a continuous period of time of about ≥30 seconds; or about ≥1 minute; or about ≥2 minutes, then the controller sounds an alarm

16. The method for modulating the dew point of a humidifier according to claim 10, wherein the adjusting of the heater plate temperature in step (M) takes place if the difference between the heater plate temperature and the target heater plate temperature is about ≥0.1° C.; or about ≥2° C.; or about ≥4° C.

17. The method according to claim 10, comprising the step of repeating steps (B) to (M); or steps (B) to (M) are repeated at least about 600 times/minute; or from about 1 times/minute to about 1000 times/minute; or from about 10 times/minute to about 800 times/minute.

18. A humidifier comprising the dew point sensor system according to claim 1.

19. A medical device comprising the dew point sensor system according to claim 1.
